(19) United States
(12) Patent Application Publication
(10) Pub. No.: US 2007/0142744 A1
(43) Pub. Date: Jun. 21, 2007

(54) Tissue sample needle and method of using same

(76) Inventor: Kevin M. Provencher, Framingham, MA (US)

Correspondence Address: BROMBERG & SUNSTEIN LLP, 125 SUMMER STREET, BOSTON, MA, 02110-1618, US

(21) Appl. No.: 11/305,817

(22) Filed: Dec. 16, 2005

(51) Int. Cl. A61B 10/00

(52) U.S. Cl. 600/562; 600/564

(57) ABSTRACT

A tissue sample needle having an outer tube and an inner tube disposed within the outer tube, the inner tube having a distal end that is provided with a snare having a plurality of deformable members extending from the inner tube. The free ends of the members are coupled directly or indirectly to the outer tube. Upon rotation of the inner tube with respect to the outer tube, the members deflect inward to reduce the effective diameter of the inner tube in the deflection zone to sever and/or hold a biopsy piece within the inner tube. After removal of the needle from a body tissue, rotating the inner tube in the opposite direction causes the members to return substantially to their original positions and thereby return the inner tube substantially to its original diameter and allow the biopsy piece to be removed or ejected from the needle.

### FIELD OF THE INVENTION

The present invention relates to devices and methods for the removal of tissue samples from a body organ.

### BACKGROUND OF THE INVENTION

In the practice of medical diagnostics, it is often necessary to perform a biopsy to remove sample of the patient's tissue for pathological study. Various devices are known in the art that are intended to make the biopsy procedure faster, more reliable, easier to perform and reduces the chance of discomfort or injury to the patient.

Certain biopsy needle devices are disclosed in the following United States patents, all of which are hereby incorporated herein by reference in their entireties: U.S. Pat. No. 5,522,398 issued on Jun. 4, 1996 to Goldenberg et al. and assigned to Medsol Corporation; U.S. Pat. No. 5,634,473 issued on Jun. 3, 1997 to Goldenberg et al. and assigned to Medsol Corporation; U.S. Pat. No. 5,843,001 issued on Dec. 1, 1998 to Goldenberg; U.S. Pat. No. 6,015,391 issued on Jan. 18, 2000 to Rishton et al.; U.S. Pat. No. 6,033,369 issued on Mar. 7, 2000 to Goldenberg; and U.S. Pat. No. 6,340,351 issued on Jan. 22, 2002 to Goldenberg. These devices have a needle comprised of an outer cannula and an inner tube with an integral snare having a coil. The inner tube is attached at its distal end to the outer cannula. Proximal to the attached portion of the inner tube is a coil. After insertion of the needle into a patient's bone with the aid of a stylet, a handle is used to rotate the inner tube relative to the outer cannula and effects a decrease in diameter of the coil portion, thereby constricting the tissue so that the tissue is restricted to cause a portion of the tissue to remain in the inner tube during withdrawal of the device. The constricting action of such a device does not necessarily cut the tissue since the snare does constrict to zero diameter, but rather the tissue is often ripped upon rearward motion of the needle. The ripping action is more efficacious for bone marrow than for soft tissue, which tends to be more elastic. For example, such a device would generally be unreliable in extracting soft tissue such as that which might be extracted in a breast biopsy.

### SUMMARY OF THE INVENTION

In accordance with one aspect of the invention there is provided a tissue sample needle comprising an outer tube having a proximal end and a distal end and an inner tube disposed within the outer tube, the inner tube comprising a tube body constituting a proximal end of the inner tube, a securing member constituting a distal end of the inner tube and coupled to the outer tube at the distal end of the outer tube, and a snare having a plurality of deformable members respectively coupled at a first end to the securing member and at a second end to the tube body. The plurality of deformable members are configured to deform inward upon rotation of the tube body relative to the securing member and the outer tube so as to effectively reduce the diameter of the inner tube within a zone of deformation for obtaining a tissue sample within the tube body.

In related embodiments, the tube body, the securing member, and the snare may be formed from a single tube, for example, by laser cutting. Alternatively, the deformable members may be separate structures that are attached to the tube body and to the securing member. Each deformable member may be configured to contact at least one other deformable member when the tube body is rotated relative to the securing member for substantially severing the tissue sample. The deformable members may be serpentine members, which may be nominally set at a forty-five degree angle with respect to a nominal axis of the tube body. The deformable members may be coils. The plurality of deformable members may comprise two to seven deformable members. The securing member may be coupled to the outer tube by laser welding. The distal end of the needle may be sharpened, for example, into a Franseen point.

In accordance with another aspect of the invention there is provided an inner tube for a tissue sample needle. The inner tube includes a tube body constituting a proximal end of the inner tube; a securing member constituting a distal end of the inner tube; and a snare having a plurality of deformable members respectively coupled at a first end to the securing member and at a second end to the tube body. The plurality of deformable members are configured to deform inward upon rotation of the tube body relative to the securing member so as to effectively reduce the diameter of the inner tube within a zone of deformation for obtaining a tissue sample within the tube body.

In related embodiments, the tube body, the securing member, and the snare may be formed from a single tube, for example, by laser cutting. Alternatively, the deformable members may be separate structures that are attached to the tube body and to the securing member. Each deformable member may be configured to contact at least one other deformable member when the tube body is rotated relative to the securing member for substantially severing the tissue sample. The deformable members may be serpentine members, which may be nominally set at a forty-five degree angle with respect to a nominal axis of the tube body. The deformable members may be coils. The plurality of deformable members may comprise two to seven deformable members.

In accordance with another aspect of the invention there is provided an inner tube for a tissue sample needle having an inner tube and an outer support structure, the inner tube comprising a tubular body constituting a proximal end of the inner tube and a plurality of deformable members respectively coupled at a first end to a support structure and at a second end to the tubular body. The plurality of deformable members configured to deform upon rotation of the tube body relative to the support structure so as to effectively reduce the diameter of the inner tube within a zone of deformation.

In related embodiments, the support structure may be an outer tube. Each deformable member may be configured to contact at least one other deformable member upon rotation of the tube relative to the support structure for substantially severing the tissue sample.

In accordance with another aspect of the invention there is provided a tissue sample needle comprising an outer tube having a proximal end and a distal end and an inner tube within the outer tube, the inner tube having a proximal end and a distal end, a snare having a plurality of deformable members disposed between two ends of the snare, one of the ends connected to the inner tube and the other of the ends coupled to the outer tube, the snare having a first and a second position, wherein in the first position, the snare has a first diameter and wherein in the second position, the snare has a second diameter smaller than the first diameter, the snare being moved from the first position to the second position by rotation of the inner tube with respect to the outer tube in a first direction and being moved from the second position to the first position by rotation in a second opposite direction.

In related embodiments, each deformable member may be configured to contact at least one other deformable member when the snare is in the second position.

In accordance with another aspect of the invention there is provided a method of obtaining a tissue sample. The method involves inserting a tissue sample needle into a body organ, the tissue sample needle including an outer tube having a proximal end and a distal end and an inner tube disposed within the outer tube, the inner tube comprising a tube body constituting a proximal end of the inner tube, a securing member constituting a distal end of the inner tube and coupled to the outer tube at the distal end of the outer tube, and a snare having a plurality of deformable members respectively coupled at a first end to the securing member and at a second end to the tube body, the plurality of deformable members configured to deform inward upon rotation of the tube body relative to the securing member and the outer tube so as to effectively reduce the diameter of the inner tube within a zone of deformation; and actuating the needle by rotating the inner tube relative to the securing member and the outer tube.

In related embodiments, the body organ may include one of breast, liver, lung, thyroid, prostate, and kidney.

### BRIEF DESCRIPTION OF THE DRAWINGS

The foregoing and advantages of the invention will be appreciated more fully from the following further description thereof with reference to the accompanying drawings wherein:

FIGS. 1-12 show the relevant components for an 11-gauge tissue sample needle, in accordance with a first exemplary embodiment of the present invention, wherein:
FIGS. 1A-1E are schematic diagrams showing an inner tube assembly 100 for an 11-gauge tissue sample needle, in accordance with an exemplary embodiment of the present invention;
FIG. 2 is a schematic diagram showing the snare 104 in greater detail;
FIGS. 3A-3C are schematic diagrams showing the inner tube housing 106 in greater detail;
FIG. 4 is a schematic diagram showing an outer tube assembly 400 for an 11-gauge tissue sample needle, in accordance with an exemplary embodiment of the present invention;
FIGS. 5A-5C are schematic diagrams showing the outer tube 402 in greater detail;
FIGS. 6A-6C are schematic diagrams showing the outer tube housing 406 in greater detail;
FIGS. 7A-7B are schematic diagrams showing a needle assembly 700 in accordance with an exemplary embodiment of the present invention;
FIG. 8 is a schematic diagram showing the distal end 702 of the needle assembly 700 in greater detail;
FIG. 9 is a schematic diagram showing the needle assembly 700 after grinding;
FIG. 10 is a schematic diagram showing the point configuration 902 in greater detail;
FIG. 11 is a schematic diagram showing a stylet assembly 1100 for an 11-gauge tissue sample needle in accordance with an exemplary embodiment of the present invention; and
FIGS. 12A-12D are schematic diagrams showing the stylet housing 1106 in greater detail;
FIGS. 13-24 show the relevant components for a 14-gauge tissue sample needle, in accordance with a second exemplary embodiment of the present invention, wherein:
FIGS. 13A-13D are schematic diagrams showing an inner tube assembly 1300 for a 14-gauge tissue sample needle, in accordance with an exemplary embodiment of the present invention;
FIG. 14 is a schematic diagram showing the snare 1304 in greater detail;
FIGS. 15A-15C are schematic diagrams showing the inner tube housing 1306 in greater detail;
FIG. 16 is a schematic diagram showing an outer tube assembly 1600 for a 14-gauge tissue sample needle, in accordance with an exemplary embodiment of the present invention;
FIGS. 17A-17B are schematic diagrams showing the outer tube 1602 in greater detail;
FIGS. 18A-18C are schematic diagrams showing the outer tube housing 1606 in greater detail;
FIGS. 19A-19B are schematic diagrams showing a needle assembly 1900 in accordance with an exemplary embodiment of the present invention;
FIG. 20 is a schematic diagram showing the distal end 1902 of the needle assembly 1900 in greater detail;
FIG. 21 is a schematic diagram showing the needle assembly 1900 after grinding;
FIG. 22 is a schematic diagram showing the point configuration 2002 in greater detail;
FIG. 23 is a schematic diagram showing a stylet assembly 2300 for a 14-gauge tissue sample needle in accordance with an exemplary embodiment of the present invention; and
FIGS. 24A-24D are schematic diagrams showing the stylet housing 2306 in greater detail; and

FIG. 25 shows some exemplary alternative snare configurations, wherein:
FIG. 25A shows an enlarged view of an exemplary snare for a 14-gauge needle having two deformable members;
FIG. 25B shows an alternative snare configuration with two deformable members, each forming a single coil;
FIG. 25C shows an alternative snare configuration with two deformable members, each forming a double coil;
FIG. 25D shows an alternative snare configuration with six deformable members; and
FIG. 25E shows an alternative snare configuration with seven deformable members.

### DETAILED DESCRIPTION OF SPECIFIC EMBODIMENTS

In embodiments of the present invention, a tissue sample needle includes a snare designed to efficiently pinch or sever tissue or tissue-like material upon actuation of the snare with a twisting action. The snare is typically tubular in shape and may be integral to the inner tube of the tissue sample needle. The snare is effective on soft tissue as well as harder tissue such as bone marrow. Efficient pinching or severing of tissue is achieved by a deformable zone of the snare having two or more deformable members that deform inwardly upon actuation. Some or all of the deformable members may contact other deformable members upon twisting in one direction to effectively reduce the inner diameter of the snare to zero. Twisting in the opposite direction will typically expand the diameter of the tube and allow the tissue to be recovered. The deformable members may be helical or serpentine in shape. There may be two, three, four, five, six, seven, or even more deformable members. The deformable members may include sharp edges that are presented to the tissue upon actuation thereby increasing the chance of severing the tissue.

In embodiments, the twisting is achieved by directly or indirectly fixing the deformable members at a distal end to a support structure and at a proximal end to a tissue-holding structure. The zone of connection between the deformable member and the support structure may be rotationally offset from the zone of connection between the deformable member and the tissue-holding structure by a number of degrees. The offset may be advantageously be chosen to be related to the number of deformable members by an amount equal to 360° divided by the number of deformable members. The support structure serves to hold the distal end of the deformable members so that the members will deform upon application of a torsion via a rotation of the tissue-holding structure relative to the support structure. The support structure is typically an outer tube, but could be another structure, such as one or more rods that will serve to hold the distal end of the deformable members to allow the application of a rotational force.

The deformable member may be directly connected or attached to the support structure. For example, the deformable members may be flexible serpentine structures that are welded directly to a tubular support structure at their distal ends. Alternately, the deformable members may be indirectly connected or attached to the support structure. In embodiments discussed in more detail below, the deformable members may be flexible serpentine structures that are coupled to a cylindrical securing member and the cylindrical securing member may be coupled to the inside of an outer tubular support structure.

The tissue holding structure is typically a proximal region of a cylindrical support inner tube, but could be any structure capable of holding a tissue sample upon actuation of the snare. The tissue-holding structure is characterized by a lumen, an interior space in which a tissue sample is held.

In certain embodiments, the support structure is an outer tube and the snare is integral to an inner tube. The needle may be manufactured from metal (e.g., 304 stainless steel), plastic, or other material, and may advantageously be both sufficiently hard enough to cut or pinch tissue with rotation in one direction, yet resilient enough to allow release of the tissue upon rotation in the opposite direction. The needle may be designed to be disposable or reusable. The zone of deformation having the deformable members may be created by the removal of material from a zone of the inner tube to create fenestrations. The material removed may form in relief two or more helical or serpentine members. Removal of the material is readily accomplished by laser cutting but alternate modes of manufacture could be employed including wire electospark discharge machining (wire EDM), chemical or photochemical etching, or other suitable technique. The device may also be made by welding together cylindrical section of tubing and deformable members, although at potentially greater expense.

In use, the tissue sample needle is first inserted into a desired tissue, such as a suspected tumor. The needle is designed to work on soft tissues like kidney, liver, lung, thyroid, prostate or breast, but embodiments will also work on harder tissues like bone marrow. Insertion of the needle may be facilitated by the use of a stylet which is typically a sharpened rod retractably inserted through the center of the needle. The stylet may be retracted and the needle subsequently pushed in further to ensure the entry of tissue into the lumen of the tissue-holding structure. Alternately, the end of the needle may be sharp enough to penetrate without the use of a stylet. After insertion, the snare is actuated by rotation in a first direction of the tissue-holding structure relative to the support structure. Since the tissue holding structure is typically internal to the needle and the support structure external, it may be advantageous to hold the external structure fixed and rotate the internal structure and thereby avoid frictional resistance and potential tissue irritation that might result if the interior structure were held fixed and the exterior structure was rotated. However, in many cases, rotation of an external support structure will accomplish the same function. In either case, actuation of the device will cause an inward deformation of the deformable members and a resulting pinching or severing of the tissue in the zone of deformation.

The needle is then withdrawn. If the tissue is pinched or partially severed, the act of withdrawal may serve to rip the tissue, allowing it to remain in the lumen of the tissue-holding structure. If the snare is not closed tightly enough, a resilient soft tissue may slip through the snare, thereby remaining in the patient. If the tissue is cleanly severed, then no such ripping or slippage will occur, leading to more reliable operation that may be less injurious to the patient. Embodiments presented herein are designed to be more reliable by creating an increased constriction and an increased chance of complete or partial cutting.

After withdrawal, the sample is typically recovered for further diagnostic or forensic analysis such as a pathological analysis for the presence of cancerous cells by cytology, histology, immunohistochemistry, messenger RNA profiling or other technique. Recovery of the sample may be accomplished by rotation of the sample-holding structure relative to the support structure in a second direction opposite to the direction of the first rotation so as to expand the inner diameter of the snare. The sample is then typically extracted by applying a force to the sample to dislodge it from the sample-holding structure. The force is typically applied with a rod, which may be the stylet, but could also be, without limitation, a squeezing, decelerating, pneumatic or hydraulic force. The same needle may be used many times on the same patient. For example, 6, 8 or even more samples may be obtained using the same needle. When compared to the coiled snare design patented by Goldenberg as listed above, embodiments of the current invention employing multiple deformable members generally allow for pinching or cutting of the sample with a smaller degree or rotation of the inner and outer tubes, thus giving a greater reliability and confidence that the device will operate without malfunction to obtain the desired number of tissue samples. In contrast to embodiments of the present invention, needles of the Goldenberg coil design cannot effectively close to an internal diameter of zero and do not allow tissue to be severed without substantial ripping.

FIGS. 1-12 show the relevant components for an 11-gauge tissue sample needle, in accordance with a first exemplary embodiment of the present invention.

FIGS. 1A-1E are schematic diagrams showing an inner tube assembly 100 for an 11-gauge tissue sample needle, in accordance with an exemplary embodiment of the present invention. As shown in FIG. 1A, the inner tube assembly 100 includes an inner tube 102 with a snare 104 located at a distal end and an inner tube housing 106 located at a proximal end. FIG. 1B is a perspective view of the inner tube assembly 100. FIG. 1C is a side view of a blank inner tube 102. FIG. 1D is cross-sectional view of the inner tube 102 showing outer diameter between approximately 0.108 and 0.110 inches and an inner diameter between approximately 0.098 and 0.101 inches. FIG. 1E is a side view of the inner tube 102 showing a wire hub 108 that is formed on the inner tube 102 approximately 0.200 inches from the proximal end to help secure the inner tube housing 106 on the inner tube 102.

FIG. 2 is a schematic diagram showing the snare 104 in greater detail. The snare 104 includes two deformable members 204 and 206 that are coupled respectively at a proximal end to tube body 202 and at a distal end to a securing member 208. The deformable members 204 and 206 are serpentine/helical in shape and are approximately 0.036 inches in width and nominally oriented at an approximately 45 degree angle with respect to the axis of the inner tube 102. The deformable members 204 and 206 are typically formed by laser cutting the inner tube 102 to remove interstitial material.

FIGS. 3A-3C are schematic diagrams showing the inner tube housing 106 in greater detail. FIG. 3A is a top view of the inner tube housing 106. FIG. 3B is an end view of the inner tube housing 106. FIG. 3C is a side view of the inner tube housing 106. The inner tube housing 106 is typically molded onto the proximal end of the inner tube 102, although it could be produced in other ways. The inner tube housing 106 includes protrusions 302 (one on each side) approximately 0.150 inches in diameter. During use of the tissue sample needle, the protrusions 302 may be used for, among other things, preventing backward displacement and rotation of the needle during a thrusting of the needle and subsequently rotating the inner tube 102 relative to an outer tube during operation of the snare 104, as discussed below.

FIG. 4 is a schematic diagram showing an outer tube assembly 400 for an 11-gauge tissue sample needle, in accordance with an exemplary embodiment of the present invention. The outer tube assembly 400 includes an outer tube 402 with an outer tube housing 406 located at a proximal end. The outer tube 402 typically includes depth indicator markings toward the distal end to aid in obtaining a tissue sample.

FIGS. 5A-5C are schematic diagrams showing the outer tube 402 in greater detail. FIG. 5A is a side view of the outer tube 402 showing the depth indicator markings at approximately one centimeter intervals along a length of the outer tube 402. FIG. 5B is cross-sectional view of the outer tube 402 showing outer diameter between approximately 0.119 and 0.121 inches and an inner diameter between approximately 0.110 and 0.114 inches. FIG. 5C is a side view of the outer tube 402 showing a wire hub 508 that is formed on the outer tube 402 approximately 0.70 inches from the proximal end to help secure the outer tube housing 406 on the outer tube 402.

FIGS. 6A-6C are schematic diagrams showing the outer tube housing 406 in greater detail. FIG. 6A is a top view of the outer tube housing 406. FIG. 6B is an end view of the outer tube housing 406. FIG. 6C is a side view of the outer tube housing 406. The outer tube housing 406 is typically molded onto the proximal end of the outer tube 402, although it could be produced in other ways. The outer tube housing 406 includes protrusions 602 (one on each side). During use of the tissue sample needle, the protrusions 602 may be used for, among other things, maintaining linear movement of the needle during a thrusting of the needle and preventing rotation of the outer tube 402 during operation of the snare 104, as discussed below.

FIGS. 7A-7B are schematic diagrams showing a needle assembly 700 in accordance with an exemplary embodiment of the present invention. The needle assembly 700 includes an inner tube assembly 100 disposed within an outer tube assembly 400. The distal end of the inner tube 102 (and, more particularly, the securing member 208) is coupled to the distal end of the outer tube 402, for example, by laser welding about a circumference of the distal end of the outer tube 402. FIG. 7A is a side view of the needle assembly 700 showing the inner tube housing 106 protruding from the proximal end of the outer tube assembly 400. FIG. 7B is a top view of the needle assembly 700, highlighting the distal end 702 of the needle assembly 700 where the inner tube 102 and outer tube 402 are laser welded.

FIG. 8 is a schematic diagram showing the distal end 702 of the needle assembly 700 in greater detail. The inner tube 102 and outer tube 402 are laser welded in area 802, which is in the area of the securing member 208 and does not interfere with the deformable members 204 and 206. As discussed below, the distal end 702 of the needle assembly 700 is ground after welding in order to form a desired point configuration (e.g., a Franseen point). The weld is typically designed to substantially follow the outline of the grinding.

FIG. 9 is a schematic diagram showing the needle assembly 700 after grinding. As mentioned above, the distal end 702 of the needle assembly 700 is ground after welding in order to form a desired point configuration 902.

FIG. 10 is a schematic diagram showing the point configuration 902 in greater detail.

FIG. 11 is a schematic diagram showing a stylet assembly 1100 for an 11-gauge tissue sample needle in accordance with an exemplary embodiment of the present invention. The stylet assembly 1100 includes a stylet 1102 and a stylet housing 1106. The stylet 1102 is typically sharpened at its distal end. The stylet assembly 1100 may be disposed within the inner tube assembly 100 during operation of the tissue sample needle to facilitate insertion of the needle into a body tissue and/or to remove a tissue sample from the inner tube 102. The stylet assembly 1100 is typically designed so that the sharpened tip protrudes from the distal end of the needle when the stylet assembly 1100 if fully inserted through the inner tube assembly 100.

FIGS. 12A-12D are schematic diagrams showing the stylet housing 1106 in greater detail. FIG. 12A is a top view of the stylet housing 1106. FIG. 12B is an end view of the stylet housing 1106. FIG. 12C is a side view of the stylet housing 1106 showing the sharpened end. FIG. 12D is a cross-sectional view of the stylet housing 1106. The stylet housing 1106 is typically molded onto the proximal end of the stylet 1102, although it could be produced in other ways. The stylet housing 1106 includes protrusions 1202 (one on each side). During use of the tissue sample needle, the protrusions 1202 may be used for, among other things, securing the stylet assembly 1100, as discussed below.

FIGS. 13-24 show the relevant components for a 14-gauge tissue sample needle, in accordance with a second exemplary embodiment of the present invention.

FIGS. 13A-13D are schematic diagrams showing an inner tube assembly 1300 for a 14-gauge tissue sample needle, in accordance with an exemplary embodiment of the present invention. As shown in FIG. 13A, the inner tube assembly 1300 includes an inner tube 1302 with a snare 1304 located at a distal end and an inner tube housing 1306 located at a proximal end. FIG. 13B is a perspective view of the inner tube assembly 1300. FIG. 13C is a side view of the inner tube 1302 showing a wire ring 1308 that is formed on the inner tube 1302 approximately 0.85 inches from the proximal end to help secure the inner tube housing 1306 on the inner tube 1302. FIG. 13D is cross-sectional view of the inner tube 1302 showing outer diameter between approximately 0.0715 and 0.0725 inches and an inner diameter between approximately 0.0635 and 0.0655 inches.

FIG. 14 is a schematic diagram showing the snare 1304 in greater detail. The snare 1304 includes two deformable members 1404 and 1406 that are coupled respectively at a proximal end to tube body 1402 and at a distal end to a securing member 1408. The deformable members 1404 and 1406 are serpentine/helical in shape and are approximately 0.011 inches in width and nominally oriented at an approximately 45 degree angle with respect to the axis of the inner tube 1302. The deformable members 1404 and 1406 are typically formed by laser cutting the inner tube 1302 to remove interstitial material.

FIGS. 15A-15C are schematic diagrams showing the inner tube housing 1306 in greater detail. FIG. 15A is a top view of the inner tube housing 1306. FIG. 15B is an end view of the inner tube housing 1306. FIG. 15C is a side view of the inner tube housing 1306. The inner tube housing 1306 is typically molded onto the proximal end of the inner tube 1302, although it could be produced in other ways. The inner tube housing 1306 includes protrusions 1502 (one on each side) approximately 0.150 inches in diameter. During use of the tissue sample needle, the protrusions 1502 may be used for, among other things, preventing backward displacement and rotation of the needle during a thrusting of the needle and subsequently rotating the inner tube 1302 relative to an outer tube during operation of the snare 1304, as described in detail below. It should be noted that the overall dimensions of the inner tube housing 1306 may be substantially the same as the dimensions of the inner tube housing 106 so that either can be used in a common tissue sample needle actuator, as discussed below.

FIG. 16 is a schematic diagram showing an outer tube assembly 1600 for a 14-gauge tissue sample needle, in accordance with an exemplary embodiment of the present invention. The outer tube assembly 1600 includes an outer tube 1602 with an outer tube housing 1606 located at a proximal end. The outer tube 1602 typically includes depth indicator markings toward the distal end to aid in obtaining a tissue sample.

FIGS. 17A-17B are schematic diagrams showing the outer tube 1602 in greater detail. FIG. 17A is a side view of the outer tube 1602 showing the depth indicator markings at approximately one centimeter intervals along a length of the outer tube 1602 and also showing a wire ring 1708 that is formed on the outer tube 1602 approximately 0.70 inches from the proximal end to help secure the outer tube housing 1606 on the outer tube 1602. FIG. 17B is cross-sectional view of the outer tube 1602 showing outer diameter between approximately 0.082 and 0.084 inches and an inner diameter between approximately 0.073 and 0.076 inches.

FIGS. 18A-18C are schematic diagrams showing the outer tube housing 1606 in greater detail. FIG. 18A is a top view of the outer tube housing 1606. FIG. 18B is an end view of the outer tube housing 1606. FIG. 18C is a side view of the outer tube housing 1606. The outer tube housing 1606 is typically molded onto the proximal end of the outer tube 1602, although it could be produced in other ways. The outer tube housing 1606 includes protrusions 1802 (one on each side). During use of the tissue sample needle, the protrusions 1802 may be used for, among other things, maintaining linear movement of the needle during a thrusting of the needle and preventing rotation of the outer tube 1602 during operation of the snare 1304, as described in detail below. It should be noted that the overall dimensions of the outer tube housing 1606 may be substantially the same as the dimensions of the outer tube housing 406 so that either can be used in a common tissue sample needle actuator, as discussed below.

FIGS. 19A-19B are schematic diagrams showing a needle assembly 1900 in accordance with an exemplary embodiment of the present invention. The needle assembly 1900 includes an inner tube assembly 1300 disposed within an outer tube assembly 1600. The distal end of the inner tube 1302 (and, more particularly, the securing member 1408) is coupled to the distal end of the outer tube 1602, for example, by laser welding about a circumference of the distal end of the outer tube 1602. FIG. 19A is a side view of the needle assembly 1900 showing the inner tube housing 1306 protruding from the proximal end of the outer tube assembly 1600. FIG. 19B is a top view of the needle assembly 1900, highlighting the distal end 1902 of the needle assembly 1900 where the inner tube 1302 and outer tube 1602 are laser welded.

FIG. 20 is a schematic diagram showing the distal end 1902 of the needle assembly 1900 in greater detail. The inner tube 1302 and outer tube 1602 are laser welded in area 2002, which is in the area of the securing member 1408 and does not interfere with the deformable members 1404 and 1406. As discussed below, the distal end 1902 of the needle assembly 1900 is ground after welding in order to form a desired point configuration (e.g., a Franseen point). The weld is typically designed to substantially follow the outline of the grinding.

FIG. 21 is a schematic diagram showing the needle assembly 1900 after grinding. As mentioned above, the distal end 1902 of the needle assembly 1900 is ground after welding in order to form a desired point configuration 2102.

FIG. 22 is a schematic diagram showing the point configuration 2002 in greater detail.

FIG. 23 is a schematic diagram showing a stylet assembly 2300 for a 14-gauge tissue sample needle in accordance with an exemplary embodiment of the present invention. The stylet assembly 2300 includes a stylet 2302 and a stylet housing 2306. The stylet 2302 is typically sharpened at its distal end. The stylet assembly 2300 may be disposed within the inner tube assembly 1300 during operation of the tissue sample needle to facilitate insertion of the needle into a body tissue and/or to remove a tissue sample from the inner tube 1302. The stylet assembly 2300 is typically designed so that the sharpened tip protrudes from the distal end of the needle when the stylet assembly 2300 if fully inserted through the inner tube assembly 1300.

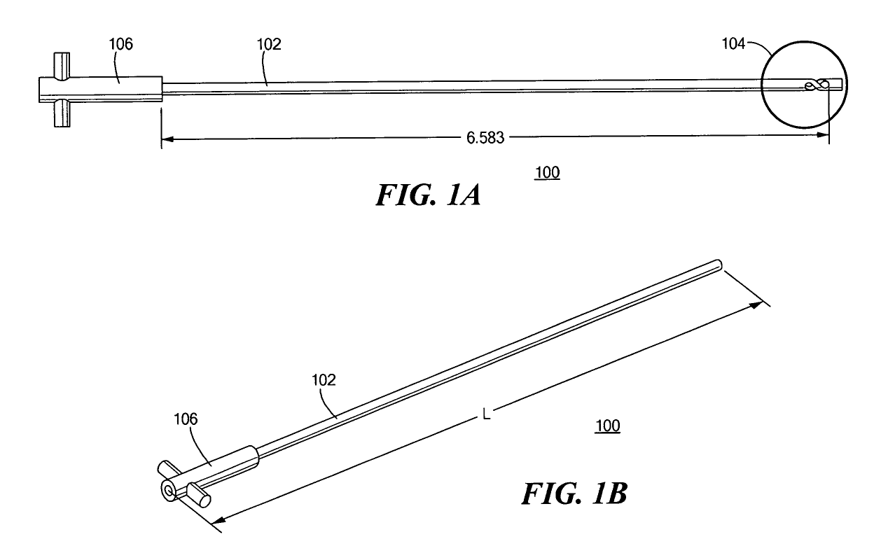

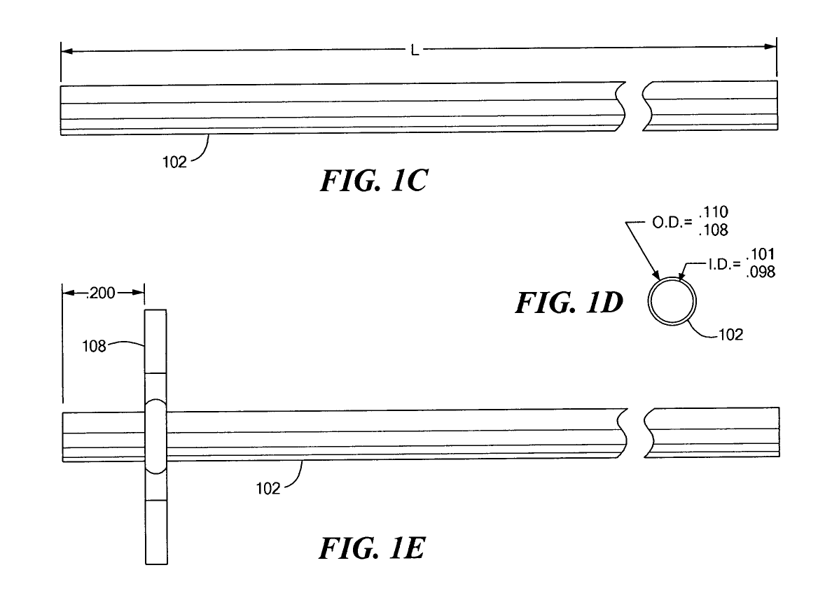

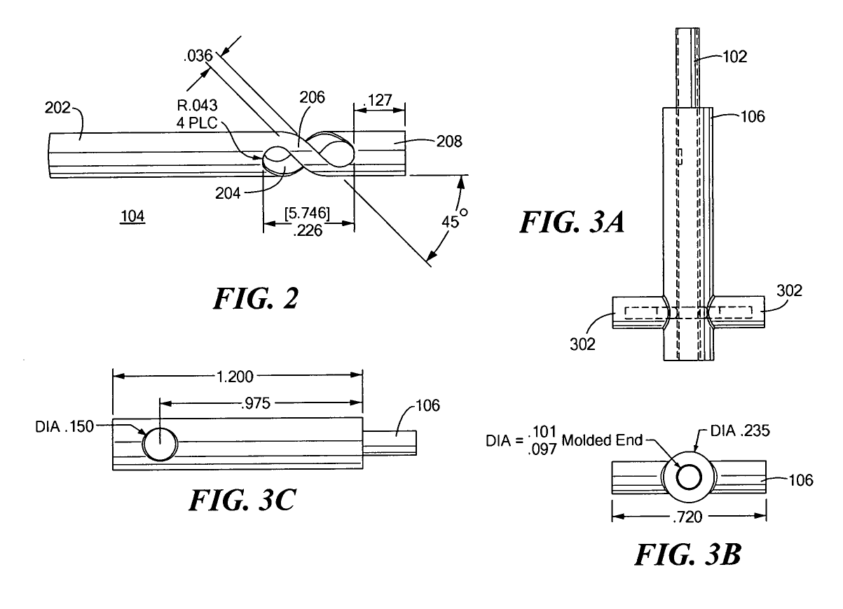

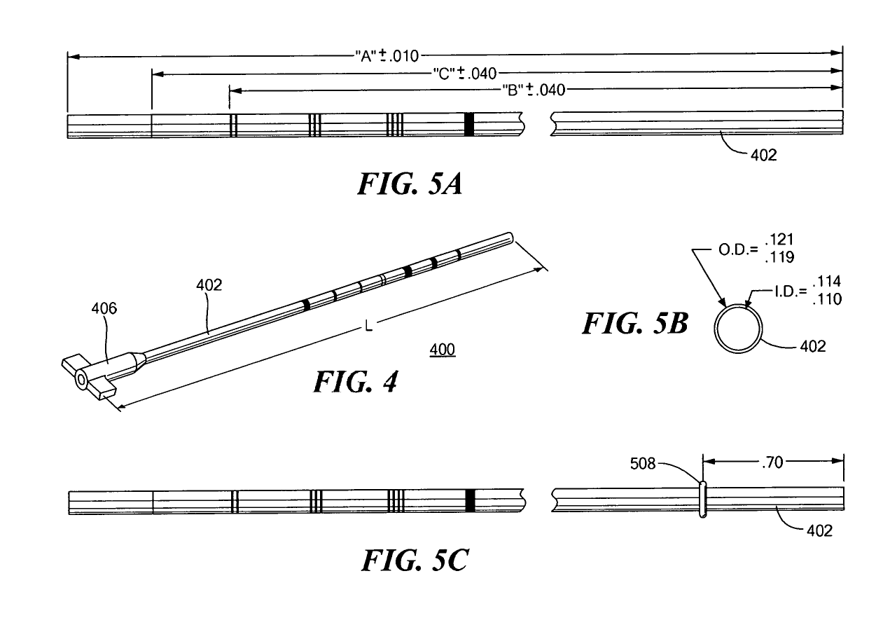

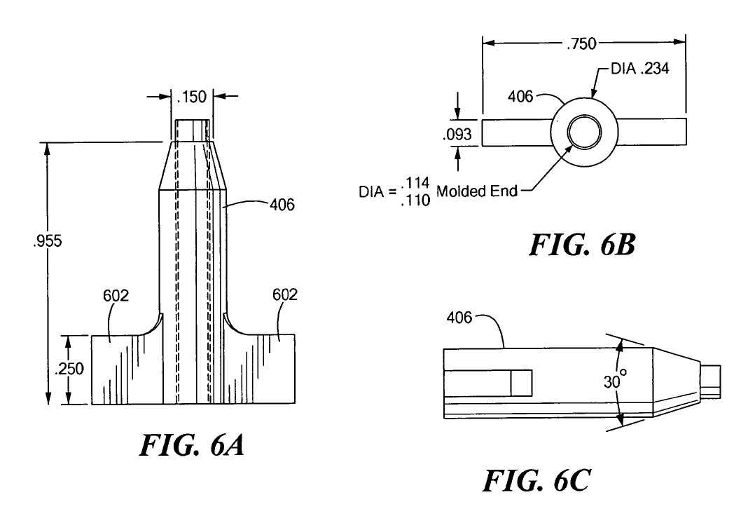

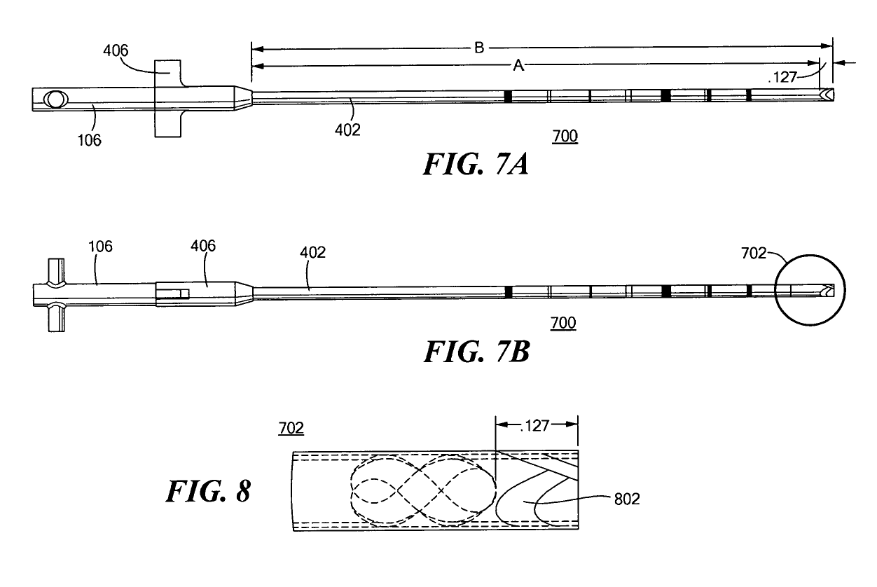

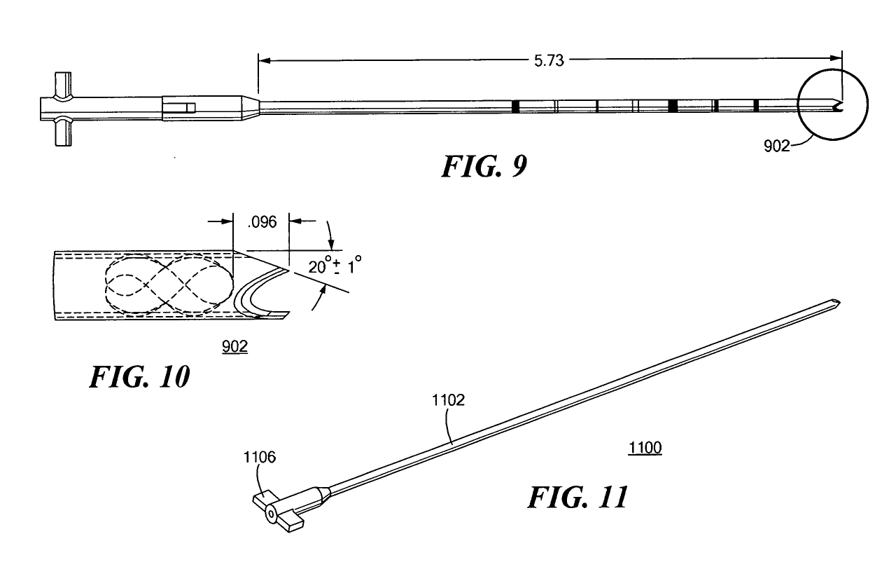

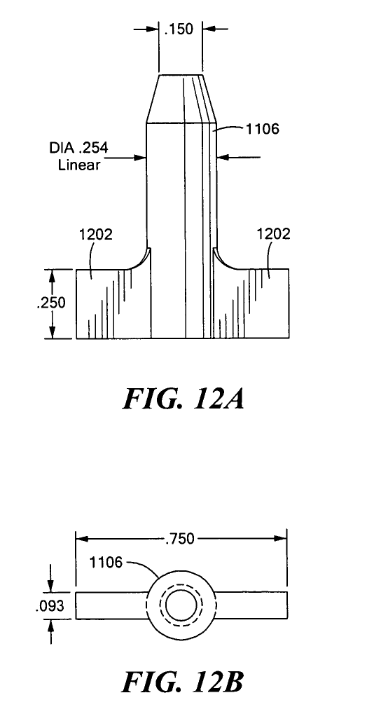

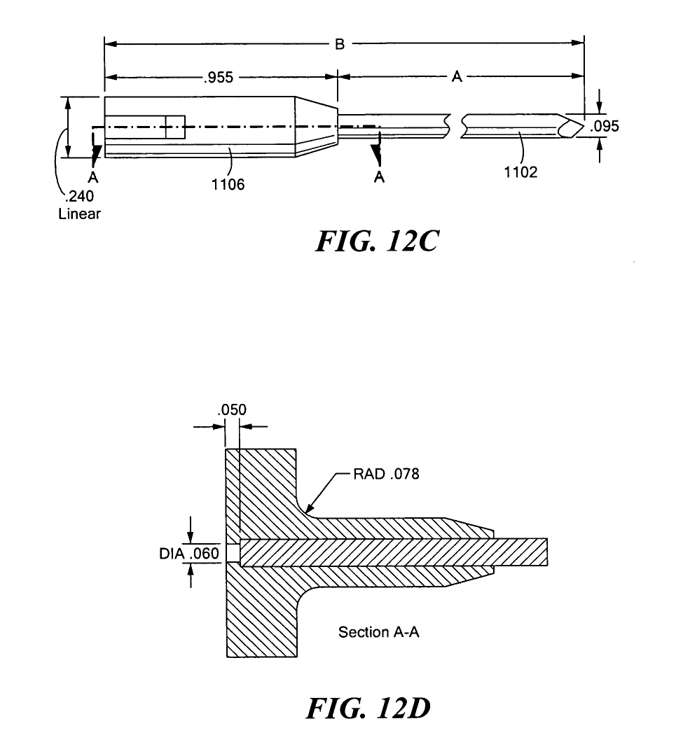

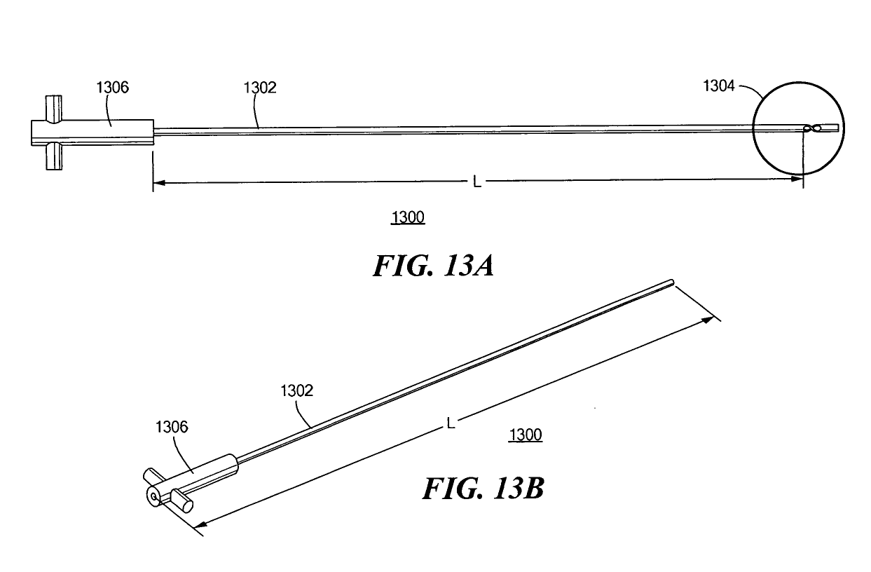

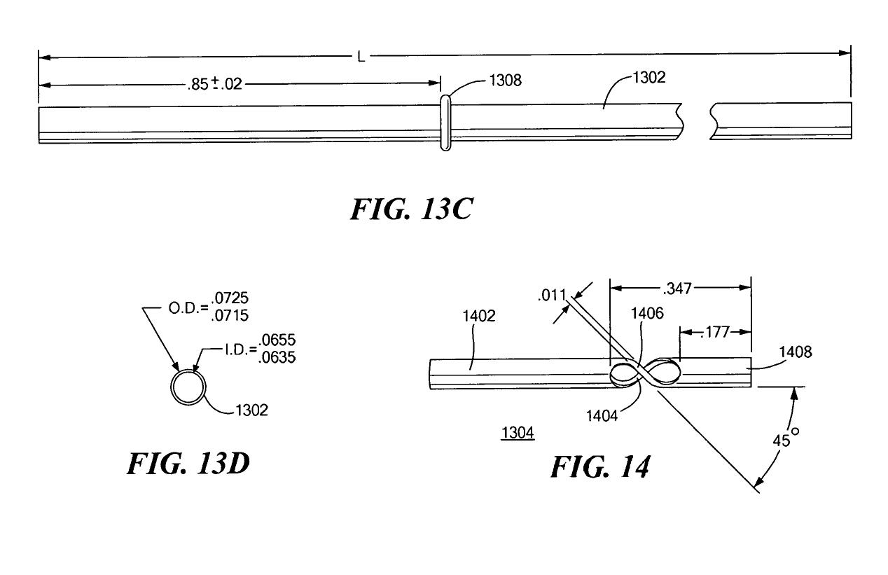

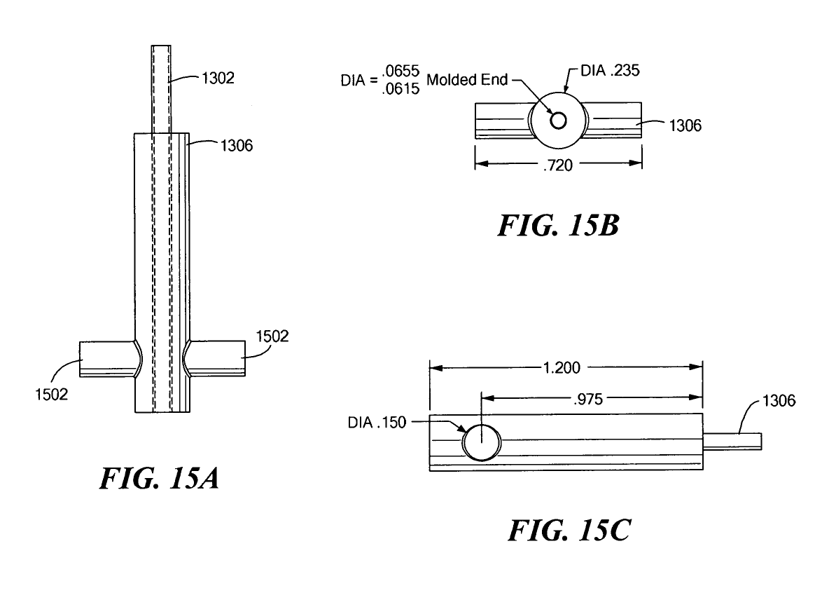

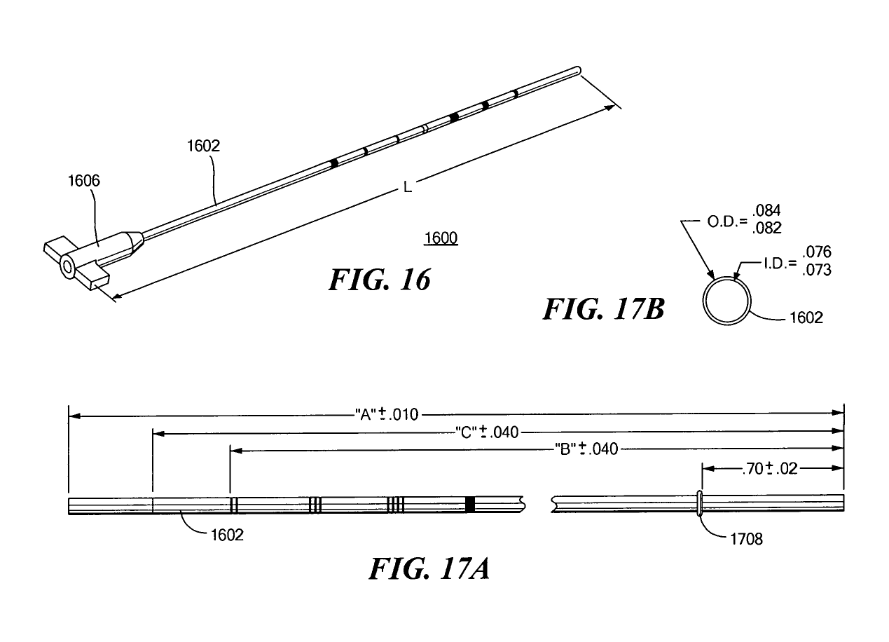

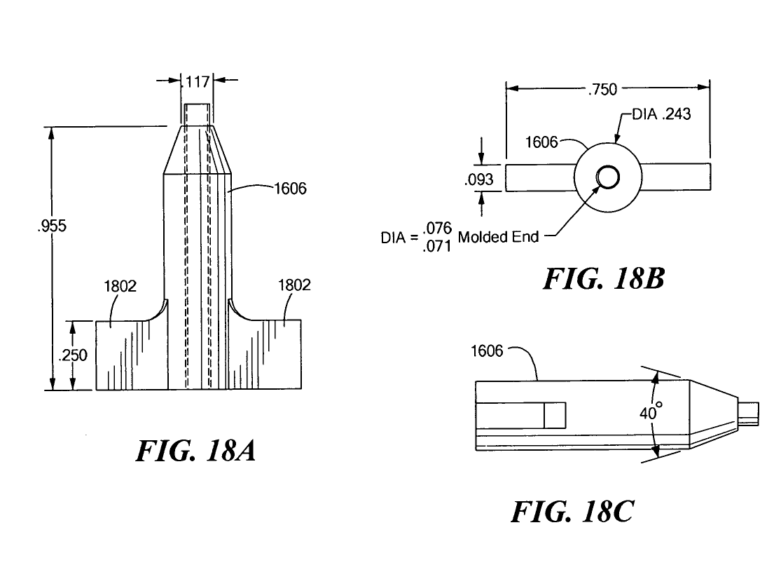

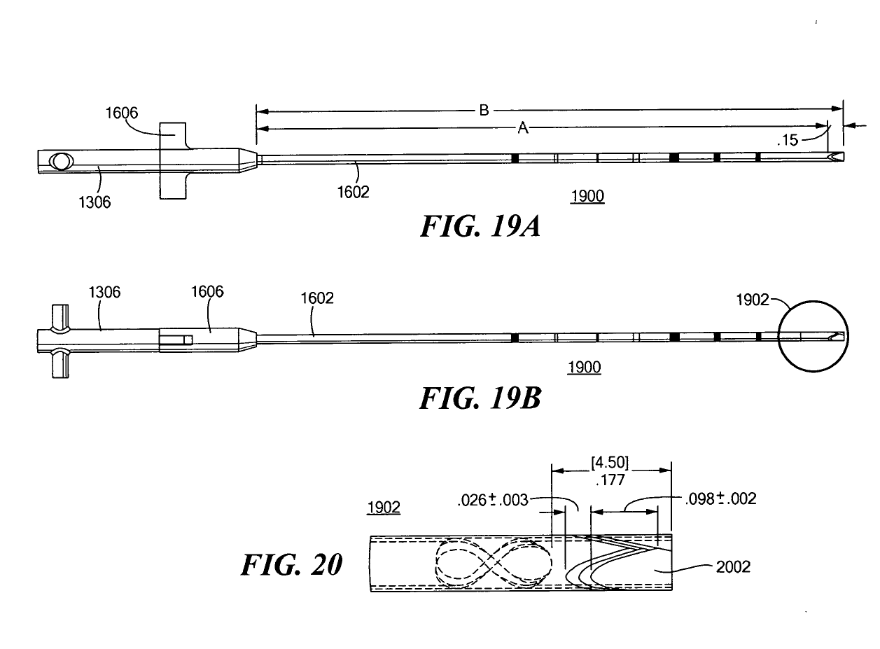

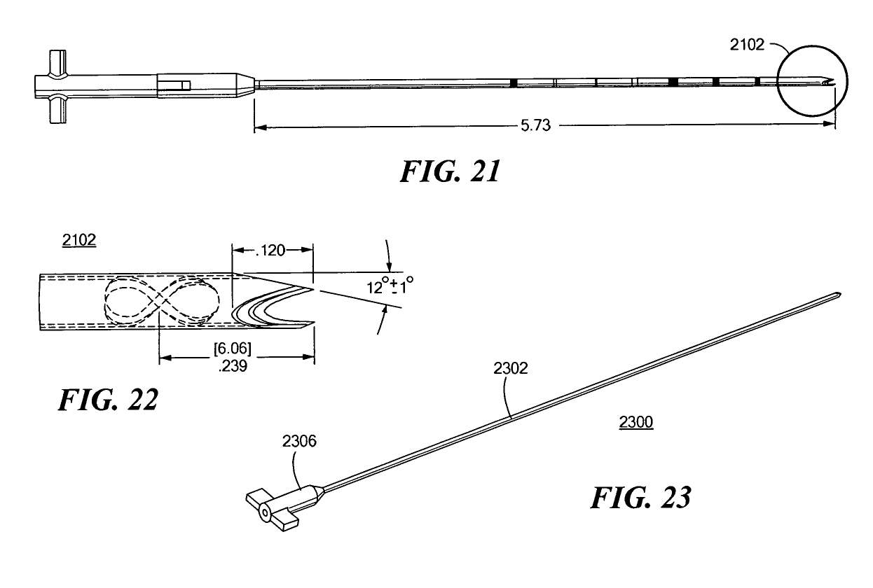

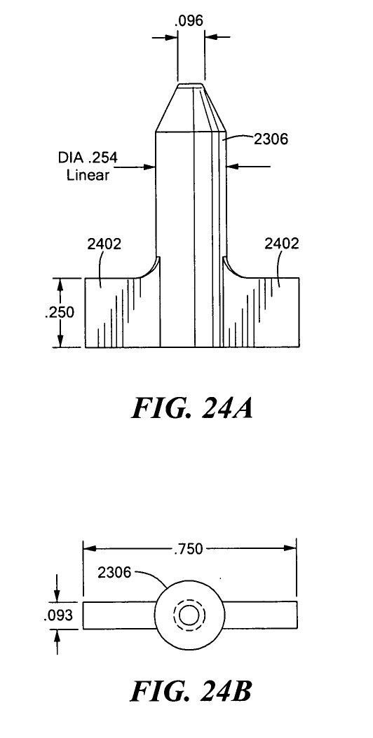

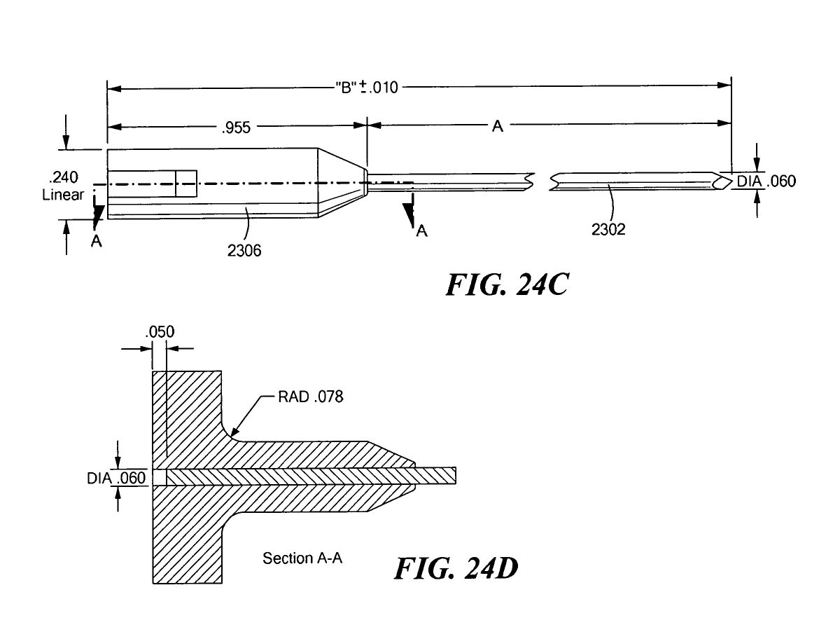

FIGS. 24A-24D are schematic diagrams showing the stylet housing 2306 in greater detail. FIG. 24A is a top view of the stylet housing 2306. FIG. 24B is an end view of the stylet housing 2306. FIG. 24C is a side view of the stylet housing 2306 showing the sharpened end. FIG. 24D is a cross-sectional view of the stylet housing 2306. The stylet housing 2306 is typically molded onto the proximal end of the stylet 2302, although it could be produced in other ways. The stylet housing 2306 includes protrusions 2402 (one on each side). During use of the tissue sample needle, the protrusions 2402 may be used for, among other things, securing the stylet assembly 2300, as described in detail below. It should be noted that the overall dimensions of the stylet housing 2306 may be substantially the same as the dimensions of the stylet housing 1106 so that either can be used in a common tissue sample needle actuator, as discussed below.

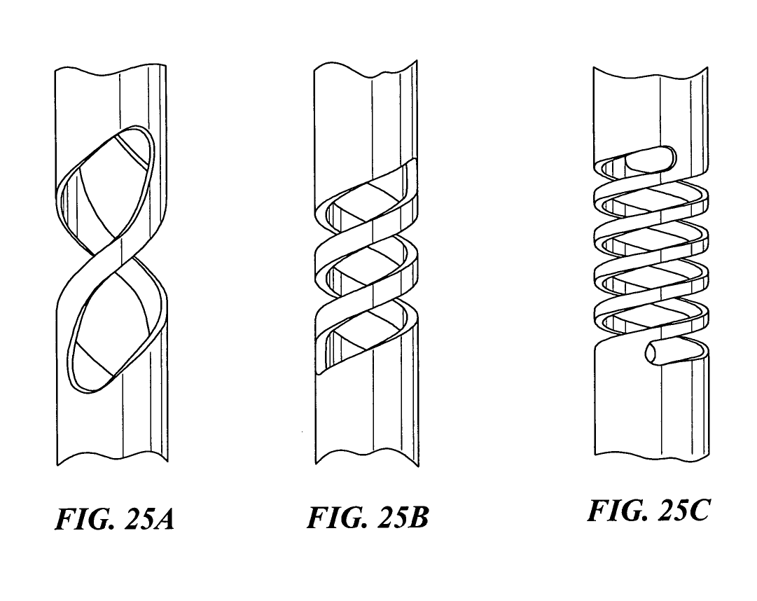

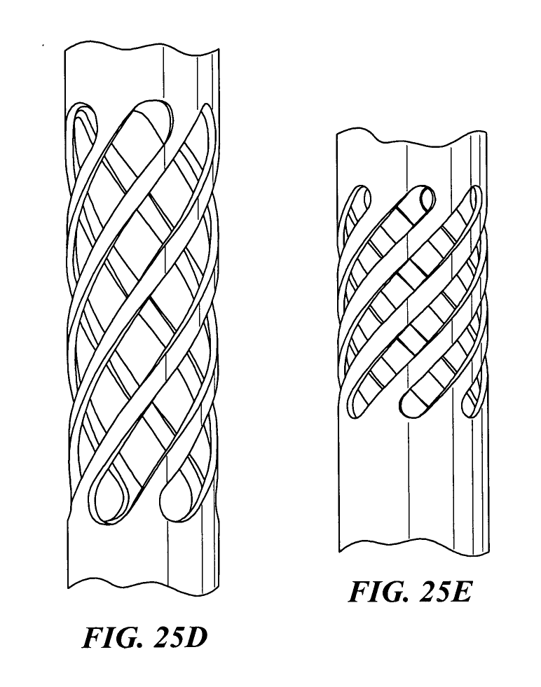

It should be understood that the present invention is not limited to any particular number, configuration, or placement of deformable members. In various alternative embodiments, the snare may include two, three, four, five, six, seven, or even more deformable members. FIGS. 25A-25E show some exemplary alternative snare configurations. FIG. 25A shows an enlarged view of an exemplary snare for a 14-gauge needle having two deformable members. FIG. 25B shows an alternative snare configuration with two deformable members, each forming a single coil. FIG. 25C shows an alternative snare configuration with two deformable members, each forming a double coil. FIG. 25D shows an alternative snare configuration with six deformable members. FIG. 25E shows an alternative snare configuration with seven deformable members. Of course, other snare configurations are possible.

It should also be understood that the present invention is not limited to any particular gauge or gauges of needles. Embodiments of the present invention can be made in 16-gauge, 18-gauge, 20-gauge, and other needle sizes. Different needle sizes may employ different snare configurations, including different numbers of deformable members and different configurations of deformable members. Even for a particular needle size, different types of needles, having different snare configurations, can be produced, for example, to meet specific requirements. For example, different types of body tissues may warrant different snare configurations.

It should be understood that the stylet assembly 1100/2300 is an optional component that can facilitate operation of the needle, for example, by facilitating insertion of the needle into a body tissue and/or facilitating ejection of a tissue sample from the needle, and should not be considered as a component of the needle itself. It should also be understood that the inner tube housing 106/1306 and the outer tube housing 406/1606 are used in certain embodiments of the invention for operation of the needle, and should not be considered as components of the needle itself. In exemplary embodiments of the present invention, the inner tube housing 106/1306, the outer tube housing 406/1606, and the stylet assembly 1100/2300 are used during operation of the needle by an actuator that automatically thrusts the needle forward into a body tissue, automatically rotates the inner tube 102/1302 relative to the outer tube 402/1606 to operate the snare, and automatically ejects the tissue sample, as described in related United States Patent Application No. XX/XXX,XXX entitled TISSUE SAMPLE NEEDLE ACTUATOR SYSTEM AND APPARATUS AND METHOD OF USING SAME, which was filed on even date herewith in the name of Kevin Provencher and Keith Orr, and is hereby incorporated herein by reference in its entirety.

The present invention may be embodied in other specific forms without departing from the true scope of the invention. The described embodiments are to be considered in all respects only as illustrative and not restrictive.

What is claimed is:

1. A tissue sample needle comprising:
an outer tube having a proximal end and a distal end; and
an inner tube disposed within the outer tube, the inner tube comprising a tube body constituting a proximal end of the inner tube, a securing member constituting a distal end of the inner tube and coupled to the outer tube at the distal end of the outer tube, and a snare having a plurality of deformable members respectively coupled at a first end to the securing member and at a second end to the tube body, the plurality of deformable members configured to deform inward upon rotation of the tube body relative to the securing member and the outer tube so as to effectively reduce the diameter of the inner tube within a zone of deformation for obtaining a tissue sample within the tube body.

2. A tissue sample needle according to claim 1, wherein the tube body, the securing member, and the snare are formed from a single tube.

3. A tissue sample needle according to claim 2, wherein the deformable members are formed by laser cutting.

4. A tissue sample needle according to claim 1, wherein the deformable members are separate structures that are attached to the tube body and to the securing member.

5. A tissue sample needle according to claim 1, wherein each deformable member is configured to contact at least one other deformable member when the tube body is rotated relative to the securing member for substantially severing the tissue sample.

6. A tissue sample needle according to claim 1, wherein the deformable members are serpentine members.

7. A tissue sample needle according to claim 6, wherein each of the serpentine members is nominally set at a forty-five degree angle with respect to a nominal axis of the tube body.

8. A tissue sample needle according to claim 1, wherein the deformable members are coils.

9. A tissue sample needle according to claim 1, wherein the plurality of deformable members comprises two to seven deformable members.

10. A tissue sample needle according to claim 1, wherein the securing member is coupled to the outer tube by laser welding.

11. A tissue sample needle according to claim 1, wherein the distal end of the needle is sharpened.

12. A tissue sample needle according to claim 11, wherein the distal end of the needle forms a Franseen point.

13. An inner tube for a tissue sample needle, the inner tube comprising:
a tube body constituting a proximal end of the inner tube;
a securing member constituting a distal end of the inner tube; and
a snare having a plurality of deformable members respectively coupled at a first end to the securing member and at a second end to the tube body, the plurality of deformable members configured to deform inward upon rotation of the tube body relative to the securing member so as to effectively reduce the diameter of the inner tube within a zone of deformation for obtaining a tissue sample within the tube body.

14. An inner tube according to claim 13, wherein the tube body, the securing member, and the snare are formed from a single tube.

15. An inner tube according to claim 14, wherein the deformable members are formed by laser cutting.

16. An inner tube according to claim 13, wherein the deformable members are separate structures that are attached to the tube body and to the securing member.

17. An inner tube according to claim 13, wherein each deformable member is configured to contact at least one other deformable member when the tube body is rotated relative to the securing member for substantially severing the tissue sample.

18. An inner tube according to claim 13, wherein the deformable members are serpentine members.

19. An inner tube according to claim 18, wherein each of the serpentine members is nominally set at a forty-five degree angle with respect to a nominal axis of the tube body.

20. An inner tube according to claim 13, wherein the deformable members are coils.

21. An inner tube according to claim 13, wherein the plurality of deformable members comprises two to seven deformable members.

22. An inner tube for a tissue sample needle having an inner tube and an outer support structure, the inner tube comprising:
a tubular body constituting a proximal end of the inner tube; and
a plurality of deformable members respectively coupled at a first end to a support structure and at a second end to the tubular body, the plurality of deformable members configured to deform upon rotation of the tube body relative to the support structure so as to effectively reduce the diameter of the inner tube within a zone of deformation.

23. A tube according to claim 22, wherein the support structure is an outer tube.

24. A tube according to claim 22, wherein each deformable member is configured to contact at least one other deformable member upon rotation of the tube relative to the support structure for substantially severing the tissue sample.

25. A tissue sample needle comprising:
an outer tube having a proximal end and a distal end;
an inner tube within the outer tube;
the inner tube having a proximal end and a distal end;
a snare having a plurality of deformable members disposed between two ends of the snare, one of the ends connected to the inner tube and the other of the ends coupled to the outer tube, the snare having a first and a second position, wherein in the first position, the snare has a first diameter and wherein in the second position, the snare has a second diameter smaller than the first diameter, the snare being moved from the first position to the second position by rotation of the inner tube with respect to the outer tube in a first direction and being moved from the second position to the first position by rotation in a second opposite direction.

26. A tissue sample needle according to claim 25, wherein each deformable member is configured to contact at least one other deformable member when the snare is in the second position.

27. A method of obtaining a tissue sample, the method comprising:
inserting a tissue sample needle into a body organ, the tissue sample needle including an outer tube having a proximal end and a distal end and an inner tube disposed within the outer tube, the inner tube comprising a tube body constituting a proximal end of the inner tube, a securing member constituting a distal end of the inner tube and coupled to the outer tube at the distal end of the outer tube, and a snare having a plurality of deformable members respectively coupled at a first end to the securing member and at a second end to the tube body, the plurality of deformable members configured to deform inward upon rotation of the tube body relative to the securing member and the outer tube so as to effectively reduce the diameter of the inner tube within a zone of deformation; and
actuating the needle by rotating the inner tube relative to the securing member and the outer tube.

28. A method according to claim 27, wherein the body organ includes one of breast, liver, lung, thyroid, prostate, and kidney.
